(19) United States
(12) Patent Application Publication
(10) Pub. No.: US 2015/0328639 A1
(43) Pub. Date: Nov. 19, 2015

(54) GENETIC TESTING KIT

(71) Applicant: GENE ONYX LIMITED, Virgin Islands (GB)

(72) Inventors: Lara BEARD, Surrey (GB); Calvin SIM, Kowloon (SG)

(21) Appl. No.: 14/651,318

(22) PCT Filed: Dec. 14, 2012
(86) PCT No.: PCT/GB2012/053147

§ 371 Date: Jun. 11, 2015

(51) Int. Cl. B01L 9/06; B01L 99/00; C12Q 1/68

(52) U.S. Cl. CPC B01L 9/06; C12Q 1/6883; B01L 99/00; C12Q 2600/148; C12Q 2600/106; C12Q 2600/156; B01L 2300/12; B01L 2300/0609

(57) ABSTRACT

A kit of parts comprising a set of two or more components and a substantially rigid box having provided on a surface thereof a visual representation of a sequence of ordered steps in a biological testing procedure. The box is provided with formations, coinciding with respective steps in said visual representation, adapted to permit insertion of said components through the surface of the box to allow the components to be supported in an upright position.

### TECHNICAL FIELD

The present invention relates to a genetic testing kit suitable for testing samples of genetic material to determine the suitability of products and ingredients for specific individuals. The invention is applicable in particular, though not necessarily, to determining the suitability of cosmetic, cosmeceutical and nutriceutical products and ingredients.

### BACKGROUND

Pharmacogenetics refers to genetic differences in metabolic pathways which can affect individual responses to drugs, both in terms of therapeutic effect as well as adverse effects. By analysing an individual's genetic material it is possible to determine the efficacy of a particular pharmaceutical component for that individual.

Single-nucleotide polymorphisms (SNPs) are the most important and basic form of variation in the genome. They are responsible for individual differences in disease susceptibility and drug response. Detection of single-nucleotide polymorphism (SNP) to evaluate particular molecule functionality in a biological pathway is used in pharmaco-genetics in order to assess the whole body health status of a patient. SNPs are identified in a patient, taking into account the pathway of interest, in order to identify susceptibility to a disease. These methods are direct sourcing of a SNP, and can be seen as responding to the question: “Is this SNP associated with a disorder or a defect?”.

It is now being recognised that genetic analysis techniques can be applied to determine the efficacy of products and ingredients that lie outside of the traditional pharmaceutical product definition. Consider for example a skin cream that might be referred to as a cosmetic or a cosmeceutical. Many factors influence the health and appearance of skin tissue including genetics, diet, hormone levels, personal hygiene, and UV exposure from the sun. Experts have long recognised a list of “active” ingredients that play a key role in skin health. These critical skin-enhancing ingredients include: a wide range of antioxidants, specific fatty acids other moisturizing agents, various vitamin, mineral co-factors and botanical elements including herbs and plant materials. The efficacy of an active ingredient relies on its ability to play a specific role in the biological pathway. This role is due to its capacity to interact with other molecules in the pathway to induce the desired response. Indeed an indirect- or direct-Response relationship between an active ingredient and its target allows the ingredient to provide the best effect of a product. As such, an analysis of an individual's genetic material, and in particular an analysis of that material for the presence or absence of one or more specific SNPs, may allow a scientific identification of a skin cream best suited to that individual.

This approach to the selection of cosmetic, cosmeceutical, nutriceutical and other related products (e.g. foods and dietary supplements) leads to the possibility of point-of-sale genetic testing. For example, a cosmetic counter in a pharmacy or department store may offer genetic testing to allow customers to identify a skin cream best suited to their skin type and needs.

Many genetic testing kits and procedures are known in the art. However, these are generally designed to be implemented in a laboratory or clinical environment, by a highly skilled technician. It is assumed that appropriate apparatus, stands, racks, pipettes, etc are available in such environments. It will be appreciated that such kits and procedures are unlikely to be suited to a point-of-sale environment, e.g. a retail premises, employing sales consultants with only limited training in the testing procedures. As such, there is a clear need for genetic testing kits that are useable in a point-of-sale environment by relatively inexperienced consultants.

### SUMMARY

It is an object of the present invention to overcome or at least mitigate the problem noted in the previous paragraph. In particular, it is an object of the present invention to provide a kit of parts for performing a biological testing procedure, for example a genetic or biochemical testing procedure, that can be used by a relatively unskilled consultant in a non-laboratory environment such as a cosmetic store or department.

According to a first aspect of the present invention there is provided a kit of parts comprising a set of two or more components and a substantially rigid box having provided on a surface thereof a visual representation of a sequence of ordered steps in a biological testing procedure. The box is provided with formations, coinciding with respective steps in said visual representation, adapted to permit insertion of said components through the surface of the box to allow the components to be supported in an upright position.

The formations provided on the box may comprise one or more of perforations, slots, and holes. The formations may be configured to provide openings into which components can be pressed.

The box may be configured to provide a closable inner space for holding at least one or more of the components prior to conducting a biological testing procedure. The box may comprise an outer sleeve and an inner compartment slidably removable from said outer sleeve for containing one or more of said components, said surface being an outer surface of the outer sleeve. The box may be of a rigid cardboard material for, e.g. reasons of cost and sustainability, but it may be made of a more durable material such as plastic, particularly if re-use is required.

The set of components may comprise one or more tubes containing, or adapted to receive, a fluid.

Said visual representation may include indicia identifying the formations, one or more of said components being labelled with indicia corresponding to the formation indicia. The visual representation may comprise markings defining said formations and lines interconnecting said markings. The visual representation may further comprise directional indicators, for example arrows or chevrons, identifying the order of steps in the sequence. The markings may comprise colours uniquely identifying respective formations, one or more of said components being marked with respective colours to assist matching of components to formations.

The kit of parts may be adapted to perform single-nucleotide polymorphisms (SNPs) detection on a sample of genetic material obtained from a client. One of said components may comprise a genetic analysis chip configured to provide an electrical output signal indicative of the presence and or absence of one or more SNPs in said sample. The genetic analysis chip being may be configured to plug into an analysis system.

The components may be suitable for use in performing a genetic testing procedure to determine, for an individual, the suitability of a plurality of cosmetic, cosmeceutical and nutriceutical products and/or ingredients.

According to a second aspect of the present invention there is provided a method of performing a genetic testing procedure using the kit of parts of any one of the preceding claims to determine, for an individual, the suitability of a plurality of cosmetic, cosmeceutical and nutriceutical products and/or ingredients. The method comprises following said sequence of ordered steps including inserting components through said formations so that they are supported by the box in an upright position, whereby biological reactions occur in these components whilst they are supported.

### BRIEF DESCRIPTION OF THE DRAWINGS

FIG. 1 illustrates schematically a box forming part of a kit of parts for performing a genetic test;

FIG. 2 illustrates a set of components forming part of a kit of parts for performing a genetic test, certain of which may be contained in the box of FIG. 1, prior to performing the genetic test;

FIG. 3 illustrates an upper surface of an outer sleeve of the box of FIG. 1, showing in particular a graphic on that surface, formations for receiving components, and components pressed through certain of the formations; and

FIG. 4 illustrates a process for performing a genetic test using the box and components of FIGS. 1 and 2.

### DETAILED DESCRIPTION

In the following discussion, reference is made to “cosmetics” and cosmetic products. This terminology is intended to cover products such as skincare products and other products, such as “nutricosmetics” (i.e. nutritional supplements that are swallowed), that are intended to somehow interact with skin and other tissue to produce a beneficial effect that may be visible or invisible (e.g. improved comfort). The terminology also covers products that may not traditionally be referred to as cosmetics, for example, moisturising creams and aftershaves.

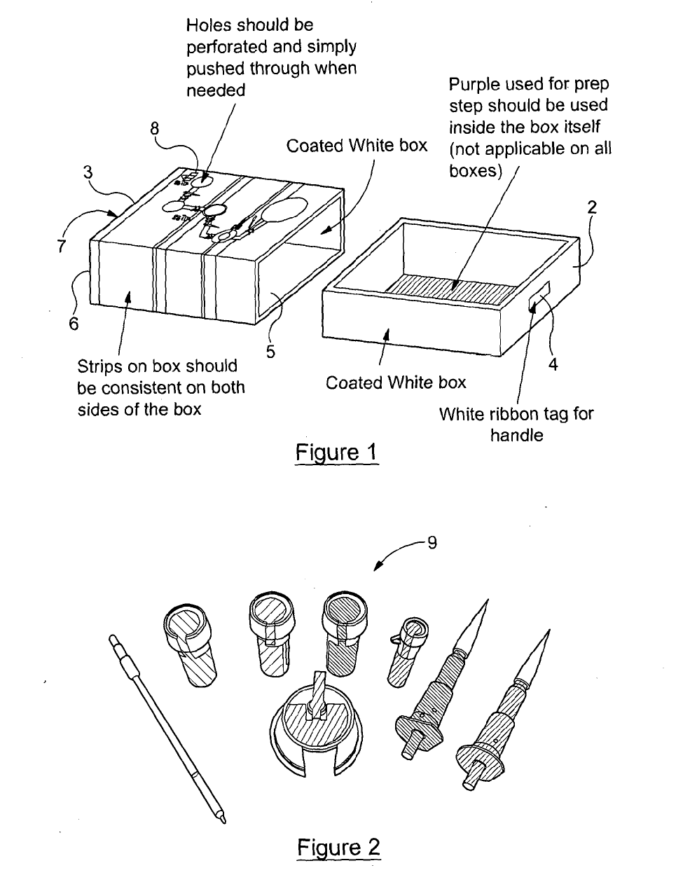

FIG. 1 illustrates a box 1 forming part of a genetic testing kit suitable for testing the genetic material of an individual. The box comprises parts, namely an inner compartment 2 open at its top, and an outer sleeve 3. The box parts are preferably formed of cardboard although other materials can be contemplated. The outer sleeve 3 is dimensioned to fit snugly around the inner compartment 2. The inner compartment 2 is provided with a tab 4 on one of its side surfaces to facilitate removal of the inner compartment from the outer sleeve. The outer sleeve is open only at one end 5, with the other end 6 being closed. The outer sleeve is both rigid and self-supporting once the inner compartment has been removed. A graphic 7 is printed over portions of the outer surface of the outer sleeve 3. This will be described in detail below, as will the inclusion and function of perforations 8 provided through the surface of the sleeve. A further (cardboard) sleeve, not shown in the figures, may also be provided to surround the outer sleeve 3 and provide protection (and further decoration) for the features provided on the surface of the outer sleeve.

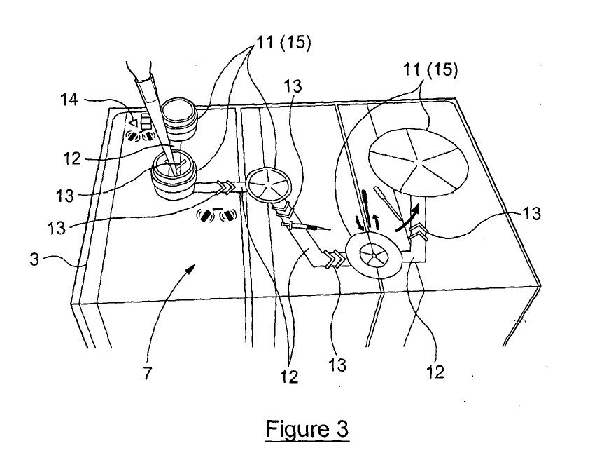

FIG. 2 illustrates a set of components 9, some of which are held within the inner compartment 2. The components are held within four separate sealed and labelled bags which are opened by the user. Prior to use, a further bag (Bag 2) is stored within a refrigerated storage area, whilst a separate “cartridge box”—not shown in the figures—holds multiple cartridge bags (Bag 3). Considering the bags further, these comprise the following:
Bag 1—rinse tube (black), saliva collection tube (green) labelled “a)”.
Bag 2 (refrigerated)—Lysis buffer tube (teal) labelled “b)”, Reagent tube (purple) labelled “d)”.
Bag 3—dock with analysis chip inserted (blue) labelled “g)”.
Bag 4—minipet A (teal), minipet B (purple).
Bag 5—pastette.

The function of the colour indicators will be clear from the following description. [NB. It will be readily apparent that the specific colours described here are merely exemplary, and that any appropriate colour schemes may be used.]

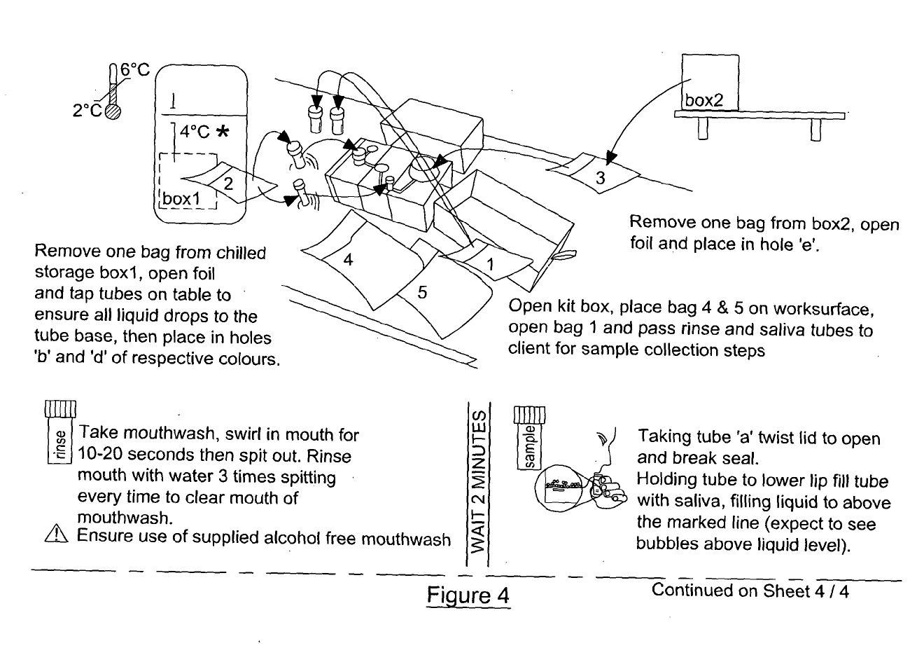

FIG. 3 shows in detail a graphic 7 printed on the upper surface of the outer sleeve 3. This graphic 7 provides a visual representation of a sequence of ordered steps in a genetic testing procedure. The graphic comprises a number of circles 11, in this case five, labelled a) to e) and interconnected in sequence by a series of lines 12 on top of which are superimposed respective chevron sets 13. The chevrons indicate the sequence in which the testing steps are to be carried out. In addition, at various points along the flow, activity indicators are printed, e.g. indicating the need for shaking of a vial (indicated by reference numeral 14), transfer of liquid, etc.

Additionally, and importantly, formations 15 or other means for breaking through the surface of the box are formed in the region of the five circles 11. These formations allow certain of the tubes and the dock to be pushed through the surface of the outer sleeve and retained therein in an upright position. The formations are substantially circular and are dimensioned to allow the openings to receive the correct tubes or the dock as appropriate. The formations 15 may be provided by, for example, perforations, cut slots, or scores.

The graphic 7 printed on the outer sleeve 3 is coloured to further indicate the sequence in which steps are to be carried out and to aid the user to match ones of the components to steps in the sequence. For example, in order to assist the user to carry out the process, the left most third of the graphic including circles labelled a) and d) may be coloured teal, the centre third, including circle c), purple, and the right most third including circle e), pale blue. The circled labelled d) straddles both the purple and light blue coloured portions of the graphic. Furthermore, specific circles are coloured to aid insertion of the tubes and docks into the respective holes.

The user, typically a sales consultant advising a client, is instructed to perform the following sequence of steps.

1) Remove inner compartment 2 from the outer sleeve 3 and remove Bags 1, 4 and 5.

2) Remove Bag 3 from cartridge box.

3) Remove Bag 2 from the fridge.

4) Break through the formations 15 in the sleeve 3.

5) Open Bag 2 and remove tubes, agitate if required,

6) Place Lysis buffer tube b) in hole b) and Reagent tube d) in hole d).

7) Open Bag 3, remove dock e) with pre inserted cartridge and insert into hole e).

8) Open Bag 1, remove the rinse tube and sample tube a) and pass to client.

9) Remove lid from rinse tube and instruct client to rinse mouth using rinse aid contained therein, spit and repeat as necessary. Discard rinse tube.

10) Remove lid from sample tube a) and instruct client to fill with saliva up to indicated fill level. Replace lid and shake. [Cells taken from around the cheek and tongue and white blood cells found in the mouth contain the DNA of interest.]

11) Stand filled sample tube a) in hole a) in outer sleeve 3. Remove lid from tube a). [The solution contained in tube a) breaks open the cells contained in the saliva to release the DNA.]

12) Remove lid from buffer tube b) currently held in stand. Open Bag 4 and remove green minipet, depress button and plunge to the base of sample tube a). Release button to withdraw saliva, remove minipet and plunge to base of buffer tube b). Then depress button to express saliva into buffer tube b).

13) Replace lids on buffer tubes a) and b). [The solution contained in tube b) allows a reaction to occur when the sample is present on the analysis chip—see below.]

14) Remove buffer tube b) from sleeve and shake. Insert buffer tube b) into hole c) in sleeve 3.

15) Remove lids from tubes b) and d), and remove the purple minipet from Bag 4. Depress button on minipet and plunge to base of buffer tube b). Release button to withdraw sample and release button. Plunge minipet to base of reagent tube d) and depress button to express sample into reagent tube d).

16) Open Bag 5 and remove pastette. Squeeze bulb of pastette and plunge to base of reagent tube d). Release bulb to draw up sample from tube, repeat action once more, this time ensuring liquid reaches the fill line etched on the pastette. d). Then remove pastette from tube d). Pull back tab to uncover opening in the dock (currently in hole e)) and insert pastette into opening. Squeeze bulb to express the sample into the dock.

17) Remove pastette and pull dock out of hole e). Then remove analysis chip from the dock.

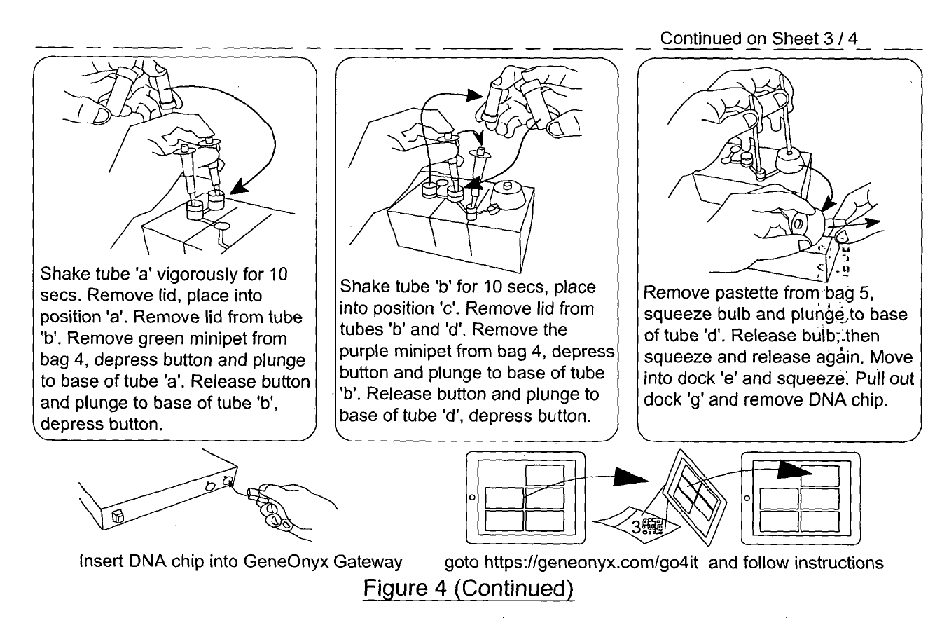

18) Insert the analysis chip into slot in the analysis system (gateway). This process is further illustrated in FIG. 4.

This completes the biochemical reaction part of the procedure. Using known structures and techniques, analysis is now performed on the sample to identify the presence of single-nucleotide polymorphisms (SNPs) within genetic material contained within the sample. The analysis system may use the results, together with additional client information such as a product type selection, e.g. skin cream, to identify ingredients/products best suited to the client's characteristics (e.g. skin type and/or metabolism). This process may utilize product databases matching SNPs to products and or ingredients. It may utilise a locally stored database or a database accessed via an Internet connection. Of course, the raw analysis data may be delivered from the gateway to some central server for analysis and product selection, with the product selection being returned to the gateway or to some other device (e.g. a wirelessly connect tablet or handheld device).

The genetic testing kit described here is designed to reduce the complexity of the testing process and to reduce the likelihood of mistakes being made, even when the consultant is relatively inexperienced and/or does no have any laboratory training.

It will be appreciated that the product and process descriptions provided above are merely exemplary. The skilled person will appreciate that various modifications may be made within the scope of the present invention. For example, all or none of the components 9 may be contained within the box 1. The box 1 may comprise a single structure, e.g. with a lid or flap that can be opened to contain components 9 if required. The box 1 may be provided as a flat pack to the point-of-sale for subsequent assembly into a structure for holding and supporting the various tubes. The graphic 7 can be varied, e.g. depending upon the number of steps involved in the reaction process. The openings provided to receive the various tubes/dock etc, may be preformed such that no break through of the box lid is required. The genetic testing kit described here may be disposable or reusable (i.e. multi-use). In the latter case, the box may have a more durable construction, e.g. it may be made of plastic.

1. A kit of parts comprising:
a set of two or more components; and
a substantially rigid box having provided on a surface thereof a visual representation of a sequence of ordered steps in a biological testing procedure, the box being provided with formations, coinciding with respective steps in said visual representation, adapted to permit insertion of said components through the surface of the box to allow the components to be supported in an upright position.

2. A kit of parts according to claim 1, wherein said formations comprise one or more of:
perforations;
slots; and
holes.

3. A kit of parts according to claim 1, wherein said formations are configured to provide openings into which components can be pressed.

4. A kit of parts according to claim 1 wherein said box is configured to provide a closable inner space for holding at least one or more of the components prior to conducting a biological testing procedure.

5. A kit of parts according to claim 1, wherein said box comprises an outer sleeve and an inner compartment slideably removable from said outer sleeve for containing one or more of said components, said surface being an outer surface of the outer sleeve.

6. A kit of parts according to claim 1, wherein said box is of a rigid cardboard material.

7. A kit of parts according to claim 1, wherein said set of components comprises one or more tubes containing, or adapted to receive, a fluid.

8. A kit of parts according to claim 1, wherein each of said visual representation includes indicia identifying the formations, one or more of said components being labelled with indicia corresponding to the formation indicia.

9. A kit of parts according to claim 1, said visual representation comprising markings defining said formations and lines interconnecting said markings.

10. A kit of parts according to claim 9, said visual representation further comprising directional indicators, for example arrows or chevrons, identifying the order of steps in the sequence.

11. A kit of parts according to claim 9, said markings comprising colours uniquely identifying respective formations, one or more of said components being marked with respective colours to assist matching of components to formations.

12. A kit of parts according to claim 1, the kit of parts being adapted to perform single-nucleotide polymorphisms (SNPs) detection on a sample of genetic material obtained from a client.

13. A kit of parts according to claim 12, wherein one of said components comprises a genetic analysis chip configured to provide an electrical output signal indicative of the presence and or absence of one or more SNPs in said sample.

14. A kit of parts according to claim 13, said genetic analysis chip being configured to plug into an analysis system.

15. A kit of parts according to claim 1, wherein said components are suitable for use in performing a genetic testing procedure to determine, for an individual, the suitability of a plurality of cosmetic, cosmeceutical and nutriceutical products and/or ingredients.

16. A method of performing a genetic testing procedure using the kit of parts of claim 1 to determine, for an individual, the suitability of a plurality of cosmetic, cosmeceutical and nutriceutical products and/or ingredients, the method comprising:
following said sequence of ordered steps including inserting components through said formations so that they are supported by the box in an upright position, whereby biological reactions occur in these components whilst they are supported.
